(19) United States
(12) Patent Application Publication
(10) Pub. No.: US 2013/0130376 A1
(43) Pub. Date: May 23, 2013

(54) SCREENING METHODS, COMPOSITIONS IDENTIFIED THEREBY, TOOLS USEFUL FOR THE IDENTIFICATION THEREOF, AND CELL POPULATIONS PRODUCED THEREBY

(71) Applicants: Naira Serobyan, San Diego, CA (US); Justin Bingham, Coronado, CA (US); Marie Zhang, San Diego, CA (US)

(72) Inventors: Naira Serobyan, San Diego, CA (US); Justin Bingham, Coronado, CA (US); Marie Zhang, San Diego, CA (US)

(21) Appl. No.: 13/695,411

(22) PCT Filed: May 2, 2011
(86) PCT No.: PCT/US11/34851

§ 371 Date: Jan. 9, 2013

Related U.S. Application Data
(60) Provisional application No. 61/330,815, filed on May 3, 2010.
(60) Provisional application No. 61/446,971, filed on Feb. 25, 2011.

(51) Int. Cl. C12N 5/095; C12N 5/071

(52) U.S. Cl. CPC C12N 5/0695
USPC 435/372.3; 506/10; 506/39

(57) ABSTRACT

In accordance with one aspect of the present invention, methods have been developed for identifying compositions which support the culture of defined cell populations. In accordance with another aspect of the present invention, methods have been developed for identifying compositions which promote differentiation of defined cell populations. In accordance with yet another aspect of the present invention, methods have been developed for identifying compositions which induce apoptosis of defined cell populations. In accordance with still another aspect of the present invention, methods have been developed for identifying compositions which promote cell senescence of defined cell populations. In accordance with still another aspect of the present invention, methods have been developed for identifying media which modulate the retardation of cell growth of defined cell subpopulation(s). In accordance with further aspects of the present invention, there are provided novel compositions identified by invention methods. Also provided are various uses of the novel compositions identified by invention methods, and novel cell populations produced employing same. In accordance with still another aspect of the present invention, methods have been developed for identifying compositions which support the culture of aberrant cell populations. In accordance with yet another aspect of the present invention, methods have been developed for identifying compositions which promote differentiation of aberrant cell populations. In accordance with still another aspect of the present invention, methods have been developed for identifying compositions which induce apoptosis of aberrant cell populations.

### RELATED APPLICATIONS

The present application claims priority from U.S. Provisional Application No. 61/330,815, filed May 3, 2010, and U.S. Provisional Application No. 61/446,971, filed Feb. 25, 2011, the contents of each of which are hereby incorporated by reference in their entirety.

### FIELD OF THE INVENTION

The present invention relates to methods for identifying compositions which support the culture of defined cell subpopulation(s). In a further aspect, the invention relates to methods for identifying compositions which promote differentiation of defined cell subpopulation(s). In a still further aspect, the invention relates to methods for identifying compositions which induce programmed cell death (apoptosis) of defined cell subpopulation(s). In yet another aspect, the invention relates to methods for identifying compositions which promote cell senescence of defined cell subpopulation(s). In still another aspect, the invention relates to methods for identifying media which modulate the retardation of cell growth of defined cell subpopulation(s). In still further aspects, the present invention relates to compositions identified by invention methods, as well as various uses thereof, and novel cell subpopulation(s) produced employing same. In yet another aspect, the present invention relates to methods for screening potentially active agents to identify those which effect a change in one or more properties of an aberrant cell population. In a still further aspect, the invention relates to methods for screening a population of aberrant cells to identify those which are susceptible to exposure to pharmacologically active agents. In yet another aspect, the invention relates to articles which facilitate carrying out the invention methods.

### BACKGROUND OF THE INVENTION

A balance between apoptosis and cell proliferation is crucial for the maintenance of homeostasis. An imbalance between cell death and proliferation may result in tumor formation. In malignant cells, apoptotic pathways are often disturbed, leading to uncontrollable growth and to resistance to anti-tumor treatment. One of the best characterized systems that trigger apoptosis is the CD95/Fas/APO-1 pathway. CD95 is expressed by hematopoietic and non hematopoietic cells including monocytes, activated lymphocytes, neutrophils and fibroblasts.

The ability to identify potentially active agents which effect a change in one or more properties (e.g., apoptosis) of an aberrant cell population (e.g., tumor cells) would be of great value in facilitating the development of new therapies.

Similarly, the ability to identify population(s) of aberrant cells (e.g., tumor cells) which are susceptible to exposure to pharmacologically active agents (e.g., antibodies, nucleic acids, and the like) and grow and/or isolate same would also be of great value in facilitating the development of new therapies.

Unfortunately, all too often cancer patients relapse. Tumors apparently vanquished by surgery and chemotherapy return months or even years later. Over the last ten years evidence has been growing that a small subset of cancer cells, sometimes referred to as Cancer Stem Cells (CSCs) or tumor initiating cells, are the driving force in tumor growth, metastasis, and recurrence. According to the cancer stem cell hypothesis, the reason tumors re-grow is because current cancer drugs kill the rapidly dividing cancer cells, but do little harm to those few cells that actually drive tumor growth. In order to successfully cure cancer (as opposed to merely treating cancer), drugs need to be developed that specifically target CSCs. Since CSCs were first identified in leukemia in 1994, they have been reported in a number of human cancers, e.g., cancers of the brain, breast, colon, pancreas, and other tissues.

Thus, yet another barrier to the development of agents which specifically target CSC's is access to these cells. In general, CSC's represent a small subset of cells within a tumor. In order to have enough CSC's at hand for such uses as disease research and drug discovery, two major hurdles must be overcome. First, one must be able to isolate such cells. Second, one must be able to propagate such cells in culture in such a manner as to maintain their tumorigenic activity and stem cell-like properties.

### SUMMARY OF THE INVENTION

In accordance with one aspect of the present invention, methods have been developed for identifying compositions which support the culture of defined cell subpopulation(s). Defined cell subpopulation(s) contemplated for use herein include:
cell subpopulation(s) which are capable of recapitulating a tumor when transplanted into an animal model;
cell subpopulation(s) that have tumorigenic activity and display stem cell-like properties;
cell subpopulation(s) that have one or more molecular markers indicative of aberrant behavior;
cell subpopulation(s) that resist drug treatment upon exposure to one or more agent employed for the treatment of hyperproliferative disorders;
cell subpopulation(s) that grow in suspension in the presence of serum-free cell culture medium containing one or more of bFGF, EGF, bovine serum albumin, leukemia inhibitory factor, Neuronal Survival Factor, insulin, human transferrin;
and the like;
   as well as combinations of any two or more thereof.

In accordance with another aspect of the present invention, methods have been developed for identifying compositions which promote differentiation of defined cell subpopulation(s) into cells that lack tumorigenic activity.

In accordance with yet another aspect of the present invention, methods have been developed for identifying compositions which induce programmed cell death (apoptosis) of defined cell subpopulation(s).

In accordance with still another aspect of the present invention, methods have been developed for identifying compositions which promote cell senescence of defined cell subpopulation(s).

In accordance with yet another aspect of the present invention, methods have been developed for identifying media which modulate the retardation of cell growth of defined cell subpopulation(s).

In accordance with further aspects of the present invention, there are provided novel compositions identified by invention methods. Also provided are various uses of the novel compositions identified by invention methods, and novel cell populations produced employing same.

Thus, invention methods enable the identification of media useful for a number of applications, e.g., supporting the culture of defined cell subpopulation(s), promoting the differentiation of defined cell subpopulation(s) into cells that lack tumorigenic activity, inducing programmed cell death (apoptosis) of defined cell subpopulation(s), promoting cell senescence of defined cell subpopulation(s), modulating the retardation of cell growth of defined cell subpopulation(s), and the like. The resulting media enable the generation of novel cell subpopulations which comprise a substantial proportion of cells having desired properties, such as, for example, cells that lack tumorigenic activity, cells which are susceptible to programmed cell death (apoptosis), cells in a state of senescence, cells which display retarded rate of cell growth, and the like.

In accordance with still another aspect of the present invention, methods have been developed for screening potentially active agents to identify those agents (and/or compositions containing same) which effect a change in one or more properties of an aberrant cell population upon exposure thereto. Invention methods are carried out employing as a support for an aberrant cell subpopulation(s) a support comprising a plurality of complex microenvironments thereon.

Aberrant cell subpopulation(s) contemplated for use herein include aberrant heart cells, aberrant hepatocytes, aberrant pancreatic cells, aberrant lung cells, aberrant brain cells, hyperproliferative cells, and the like.

In accordance with yet another aspect of the present invention, the ability of the well characterized fas receptor agonist IgM antibody to drive Jurkat T cell lymphoma cells immobilized on an extra cellular matrix substrate is demonstrated.

In accordance with still another aspect of the present invention, methods have been developed for screening a population of aberrant cells to identify those cell population(s) which are susceptible to exposure to one or more pharmacologically active agents. Invention methods are carried out employing as a support for the one or more pharmacologically active agents (or the aberrant cell subpopulation(s) being tested) a support comprising a plurality of complex microenvironments thereon.

In accordance with yet another aspect of the present invention, methods have been developed for identifying compositions which induce programmed cell death (apoptosis) of an aberrant cell subpopulation(s).

In accordance with still another aspect of the present invention, methods have been developed for identifying population(s) of aberrant cells (e.g., tumor cells) which are susceptible to exposure to pharmacologically active agents (e.g., antibodies, nucleic acids, and the like), as well as methods for the growth and/or isolation of such cell populations.

In accordance with further aspects of the present invention, there are provided articles which facilitate carrying out the invention methods. Also provided are various uses of invention articles.

### BRIEF DESCRIPTION OF THE FIGURES

FIG. 1 is an exemplary configuration of array slides employed in the practice of the present invention.

FIG. 2 is an exemplary method for fabrication of array slides for use in the practice of the present invention.

FIG. 3 illustrates the identification of components that support Jurkat cell attachment to supports comprising a plurality of complex microenvironments thereon as contemplated for use in the practice of the present invention (see Example 9).

FIG. 4 is a plot of fluorescence intensity of goat anti mouse secondary IgM conjugated to FITC when exposed to anti CD95 printed in the presence of fibronectin on hydrogel in 400 μm spots (see Example 9).

FIG. 5 illustrates that Jurkat cells could not maintain attachment to fibronectin in the presence of anti CD95 IgM monoclonal antibody (MAB). Jurkat cells were incubated for 48 hrs in the presence of anti CD95. Cell attachment to spots was observed to be dose dependent.

FIG. 6 illustrates the increase in Caspase 3 signal (within a 24 hr time period) when Jurkat cells are immobilized to fibronectin in the presence of immobilized anti CD95. This Caspase 3 activity is directly correlated to the fas mediated activated cell death pathway.

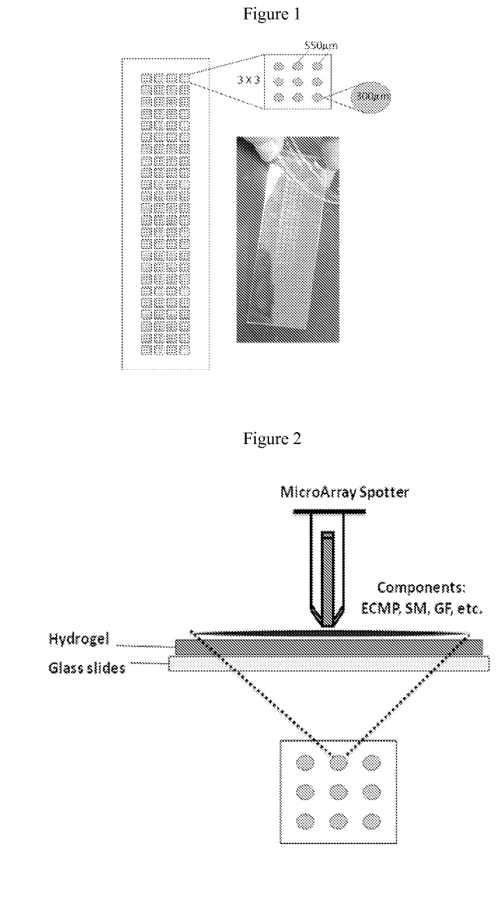

### DETAILED DESCRIPTION OF THE INVENTION

In accordance with the present invention, there are provided methods for identifying media for the short- and/or long-term in vitro culture of cell subpopulation(s) as defined herein, said method comprising:
creating a plurality of complex microenvironments, wherein each complex microenvironment comprises a plurality of components,
screening said cell subpopulation(s) against said plurality of complex micro environments, and
selecting those complex microenvironments which facilitate short-term and/or long-term in vitro culture of said cell subpopulation(s).

Defined cell populations contemplated for use herein include:
cell population(s) which are capable of recapitulating a tumor when transplanted into an animal model,
cell population(s) that have tumorigenic activity and display stem cell-like properties;
cell population(s) that have one or more molecular markers indicative of aberrant behavior;
cell population(s) that resist drug treatment upon exposure to one or more agent employed for the treatment of hyperproliferative disorders; and
cell population(s) that grow in suspension in the presence of serum-free cell culture medium containing one or more of bFGF, EGF, bovine serum albumin, leukemia inhibitory factor, Neuronal Survival Factor, insulin, human transferrin, and the like;
   as well as combinations of any two or more thereof.

As used herein, the phrase “cell population(s) which are capable of recapitulating a tumor when transplanted into an animal model” refers to the ability of target cell population(s) (which are typically derived from tumors) to promote tumor formation, for example, when transplanted into an animal model.

As used herein, the phrase “cell population(s) that have one or more molecular markers indicative of aberrant behavior” refers to cells expressing such markers as CD44+/CD24−, CXCR4+, CD133+, CD138−, CD20, alpha2beta1+, CD44+, EpCam+, Cd166+, LGR5, CD24+, and the like, as well as combinations of any two or more thereof.

As used herein, the phrase “cell population(s) that resist drug treatment upon exposure to one or more agent employed for the treatment of hyperproliferative disorders” refers to cells that are resistant to agents such as Arabinosylcytosine (ARA-C), Cytarabine, Bleomycin, Busulfan, Capecitabine, Carboplatin, Carmustine, Chlorambucil, Cisplatin, Cyclophosphamide, Dacarbazine, Daunorubicin, Docetaxel, Doxorubicin, Epirubicin Etoposide, Fludarabine, 5Fluorouracil, Gemcitabine, Hydroxyurea, Idarubicin, Ifosfamide, Irinotecan, Lomustine, Mechlorethamine, Melphalan, 6-Mercaptopurine (6-MP), Methotrexate, Mitomycin CMitoxantrone, Oxailplatin, Paclitaxel, Streptozocin, Temozolomide, 6-Thioguanine, Topotecan, Vinblastine, Vincristine, Vindesine, Vinorelbine, Alemtuzumab, Bevacizumab, Gemtuzumab, Ibritumomab, Rituximab, Tositumomab, Trastuzumab, Aldesleukin, IL-2, Alpha Interferon, Imiquimod, Lenalidomide, Anastrozole, Bicalutamide, Exemestane, Flutamide, Fulvestrant, Letrozole, Megestrol, Raloxifene, Tamoxifen, Toremifene, and the like.

As used herein, the phrase “cell population(s) that grow in suspension in the presence of serum-free cell culture medium containing one or more of bFGF, EGF, bovine serum albumin, leukemia inhibitory factor, Neuronal Survival Factor, insulin, human transferrin, and the like” refers to cells that grow in the presence of serum-free cell culture medium containing one or more of bFGF, EGF, bovine insulin, and human transferring, and which grow as spheroids in suspension culture, and which are further capable of differentiating into adherent cells in the presence of fetal bovine serum.

In accordance with another aspect of the present invention, there are provided methods for identifying media for the short-and/or long-term in vitro culture of cell subpopulation(s) as defined herein, said method comprising:
screening said cell population(s) against a plurality of complex microenvironments, wherein each complex microenvironment comprises a plurality of components, one or more of which closely resemble or mimic component(s) found in the in vivo environment in which the cell normally resides, or in the in vivo environment of a species homologous to the species from which said cell population(s) were obtained, and
selecting those microenvironments which facilitate short- and/or long-term culture of said cell population(s).

As employed herein, components which “closely resemble” components which are found in the in vivo environment of a species homologous to the species from which the cell population(s) were obtained are components which are structurally and/or functionally substantially similar to such components (e.g., analogs and/or homologs thereof).

As employed herein, components which “mimic” components which are found in the in vivo environment of a species homologous to the species from which the cell population(s) were obtained are components which impart substantially the same functional properties as the components which are found in the in vivo environment of interest.

In accordance with still another aspect of the present invention, there are provided methods for identifying media for the short-and/or long-term culture of cell subpopulation(s) as defined herein, said method comprising selecting those microenvironments which facilitate short- and/or long-term culture said cell population(s) when said cell population(s) are screened against a plurality of complex microenvironments,

wherein each complex microenvironment comprises a plurality of components, one or more of which closely resemble component(s) found in the in vivo environment in which the cell subpopulation(s) normally reside(s), or in the in vivo environment of a species homologous to the species from which said cell population(s) were obtained.

As used herein, reference to “homologous species” embraces species which are from the same family as the species of interest, e.g., primates, canines, felines, bovine, rodents, and the like are considered to be homologous to humans. Thus, one may look to mice, rats, monkeys, and the like, for component(s) found in the in vivo environment in which the cell subpopulation(s) of interest normally reside(s).

Reference herein to “homologous species” also embraces any organism which displays at least 30% sequence similarity (relative to the species of interest) at the protein level. In certain embodiments of the present invention, the term “homologous species” embraces any organism which displays at least 40% sequence similarity (relative to the species of interest) at the protein level. In certain embodiments of the present invention, the term “homologous species” embraces any organism which displays at least 50% sequence similarity (relative to the species of interest) at the protein level. In certain embodiments of the present invention, the term “homologous species” embraces any organism which displays at least 60% sequence similarity (relative to the species of interest) at the protein level, at least 70% sequence homology, or at least 80% sequence homology.

Media identified herein can be employed for a variety of applications, e.g., to propagate novel cell lines that have tumorigenic activity, cell lines that lack tumorigenic activity, cells which are susceptible to programmed cell death (apoptosis), cells in a state of senescence, cells which display retarded rate of cell growth, and the like. Such cell lines can be isolated in a variety of ways, e.g., by comparison to a replicate of the initial microenvironment and retrieval of the cells of interest.

Media identified employing invention methods typically comprise a plurality of components which, when taken together, are sufficient to support the short- and/or long-term in vitro culture of specific cell subpopulation(s), as defined herein. Such media include formulations which provide a suitable matrix for the short- and/or long-term in vitro culture of specific cell subpopulation(s).

As readily recognized by those of skill in the art, many of the various components contemplated for preparation of test media described herein need not be introduced in active form; instead, such components may be introduced in precursor form (i.e., as a chemically protected species (e.g., a prodrug), a physically protected species (e.g., coated particulate material), or the like).

In a particular embodiment of the present invention, one or more of the plurality of components which comprise each complex microenvironment closely resemble component(s) found in the in vivo environment in which said cell subpopulation(s) normally reside(s), or in the in vivo environment of a species homologous to the species from which said cell population(s) normally reside. One of skill in the art need only consult the scientific literature to identify candidate components to be tested employing the invention methodology. Minimal components include extracellular matrix proteins (ECMPs) and/or growth factors that have been shown to play a role in the viability of the subpopulation of interest.

Each of the microenvironments contemplated for use herein comprise a multi-factorial array of at least two or more components selected from the group consisting of extracellular matrix proteins or components thereof, cellular adhesion molecules, monosaccharides, oligosaccharides, polysaccharides, glycoproteins, proteoglycans, non-proteoglycan polysaccharides, cell communication molecules, complex carbohydrates, lipids, vitamins and metabolites thereof, naturally occurring low molecular weight biologically active molecules, synthetic low molecular weight biologically active molecules, synthetic polymers, biopolymers, antibodies, nucleic acids, inorganic salts, media supplements, and the like. As readily recognized by those of skill in the art, biologically active components may fit into more than one of the categories set forth above, e.g., growth factors may also be considered to be signaling molecules.

In certain aspects of the invention, each of the microenvironments contemplated for use herein comprise a multi-factorial array of three or more components selected from the various components set forth above.

In certain aspects of the invention, each of the microenvironments contemplated for use herein comprise a multi-factorial array of four or more components selected from the various components set forth above.

In certain aspects of the invention, each of the microenvironments contemplated for use herein comprise a multi-factorial array of five or more components selected from the various components set forth above.

In certain aspects of the invention, each of the microenvironments contemplated for use herein comprise a multi-factorial array of six or more components selected from the various components set forth above.

In certain aspects of the invention, each of the microenvironments contemplated for use herein comprise a multi-factorial array of seven or more components selected from the various components set forth above.

In certain aspects of the invention, each of the microenvironments contemplated for use herein comprise a multi-factorial array of eight or more components selected from the various components set forth above.

In certain aspects of the invention, each of the microenvironments contemplated for use herein comprise a multi-factorial array of nine or more components selected from the various components set forth above.

In certain aspects of the invention, each of the microenvironments contemplated for use herein comprise a multi-factorial array of ten or more components selected from the various components set forth above.

Thus, any one micro-environment will likely contain less than all of the various components referred to above, but at least one representative component referred to above will be represented in at least one of the micro-environments of a given multi-factorial array.

As readily recognized by those of skill in the art, the number of components combined to create a given microenvironment can be widely varied, as can the relative amounts of the various components contemplated for use herein to prepare the multi-factorial array. An exemplary population of microenvironments can be generated by creating various optional combinations of the components contemplated for use herein, as illustratively set forth in the following table, wherein “+” indicates a component is present (and ++ or +++ indicate the presence of a higher relative amount of such component, relative to a component which is merely “present”); and “−” indicates a component is not present in the particular microenvironment).

### Microenvironment

| Component | 1 | 2 | 3 | 4 | 5 | 6 | 7 | 8 | 9 | 10 |
|---|---|---|---|---|---|---|---|---|---|---|
| Extracellular matrix | +++ | +++ | +++ | ++ | ++ | ++ | + | + | + | − |
| proteins | | | | | | | | | | |
| Cellular adhesion | − | + | + | + | ++ | ++ | ++ | +++ | +++ | +++ |
| molecules | | | | | | | | | | |
| Saccharides | + | − | + | − | + | − | + | − | + | − |
| Glycoproteins | − | + | − | + | − | + | − | + | − | + |
| Proteoglycans | + | − | + | − | + | − | + | − | + | − |
| Cell communication | − | + | − | + | − | + | − | + | − | + |
| molecules | | | | | | | | | | |
| Complex | + | − | + | − | + | − | + | − | + | − |
| carbohydrates | | | | | | | | | | |
| Lipids | − | + | − | + | − | + | − | + | − | + |
| Vitamins | + | − | + | − | + | − | + | − | + | − |
| Biopolymers | − | + | − | + | − | + | − | + | − | + |
| Antibodies | + | − | + | − | + | − | + | − | + | − |
| Nucleic acids | − | + | − | + | − | + | − | + | − | + |
| Inorganic salts | + | − | + | − | + | − | + | − | + | − |
| Media supplements | − | + | − | + | − | + | − | + | − | + |

As used herein, the term “extracellular matrix proteins” refers to structural proteins which provide structural integrity to cells. Exemplary extracellular matrix proteins contemplated for use herein, or functional components thereof, include collagen, fibronectin, laminin, elastin, vitronectin, tenascin, decorin, and the like, as well as combinations of any two or more thereof.

Exemplary collagens contemplated for use herein include Type I fibrillar collagen, Type II fibrillar collagen, Type III fibrillar collagen, Type V fibrillar collagen, Type XI fibrillar collagen, Type IX facit collagen, Type XII facit collagen, Type XIV facit collagen, Type VIII short chain collagen, Type X short chain collagen, Type IV basement membrane collagen, Type VI collagen, Type VII collagen, Type XIII collagen, and the like, as well as combinations of any two or more thereof.

Exemplary cellular adhesion molecules (CAM) contemplated for use herein, or components thereof, include members of the immunoglobulin superfamily (IgSF CAMs), the integrins, the cadherins, the selectins, the lymphocyte homing receptors, and the like, as well as combinations of any two or more thereof.

Exemplary mono- and oligosaccharides contemplated for use herein, or components thereof, include trioses, tetroses, pentoses, hexoses, heptoses, octoses, nonoses, sucrose, lactose, maltose, trehalose, turanose, cellobiose, raffinose, melezitose, malotriose, acarbose, stachyose, and the like, as well as combinations of any two or more thereof.

Exemplary glycoproteins contemplated for use herein, or components thereof, include proteoglycans and non-proteoglycan polysaccharides, and the like, as well as combinations of any two or more thereof. Exemplary glycoproteins include heparin, heparan sulfate, chondroitin sulfate, dermatan sulfate, keratan sulfate, hyaluronic acid, perlecan, aggrecan, versican, decorin, biglycan, fibromodulin, lumican, and the like, as well as combinations of any two or more thereof.

Cell communication molecules contemplated for use in the practice of the present invention include growth factors, hormones, signaling molecules, cytokines, and the like, as well as combinations of any two or more thereof.

Growth factors contemplated for use herein include any substance capable of stimulating cellular growth, proliferation and/or differentiation, typically a protein or a steroid hormone. Presently preferred growth factors are endogenous to the species of organism from which the desired cell population is obtained, or endogenous to a species homologous to the species from which the desired cell population is obtained, as well as combinations of any two or more thereof. Growth factors are sometimes referred to in the art as cytokines, although as used herein, cytokines are but a subset of the compounds contemplated for use herein.

Exemplary growth factors include angiopoietin-1, angiopoietin-2, brain-derived neurotrophic factor (BDNF), one or more members of the BMP signaling family, one or more members of the Wnt family, osteopontin, one or more members of the epidermal growth factor (EGF) family, one or more members of the epidermal growth factor-CriptoFRL1Cryptic (EGF-CFC) family, EPO, Eotaxin, one or more members of the fibroblast growth factor (FGF) family, FLT-3 ligand, one or more members of the hepatocyte growth factor (HGF) family, one or more members of the insulin growth factor (IGF) family, platelet-derived growth factor, sonic hedgehog, one or more members of the transforming growth factor (TGF), family, TPO, one or more members of the vascular endothelial growth factor (VEGF) family, PIGF, Rantes, stromal cell-derived factor (SDF), Granulocyte-colony stimulating factor (G-CSF), granulocyte-macrophage colony stimulating factor (GM-CSF), growth differentiation factor-9 (GDF9), hepatoma derived growth factor (HDGF), myostatin (GDF-8), neurotrophins such as nerve growth factor (NGF), platelet-derived growth factor (PDGF), amine-derived hormones, peptide hormones, lipid and phospholipid-derived hormones, the IL-2 subfamily,the interferon (IFN) subfamily, the IL-10 subfamily, the IL-1 family, the IL-17 family, and the like, as well as combinations of any two or more thereof.

Exemplary hormones contemplated for use herein include steroids, retinoic acid, thyroid hormone, vitamin D3, insulin, parathyroid hormone, luteinizing hormone releasing factor (LHRH), alpha and beta seminal inhibins, human growth hormone, and the like.

Exemplary cytokines contemplated for use herein include GM-CSF, G-CSF, M-CSF, one or more members of the interferon family, one or more members of the interleukin family, one or more members of the TNF family, one or more members of the transferrin family, insulin, one or more members of the human growth hormone (HGH) family, one or more prostanoids, one or more members of the prostaglandin hormone family, GRO/KC/CINC chemokines, kallikrein, oncostatin, osteoprotegerin, one or more members of the sphingosine family, one or more MCP/MCAF chemokines, MIG, macrophage inflammatory protein (MIP) chemokines, and the like, as well as combinations of any two or more thereof.

Exemplary signaling components contemplated for use herein include any signaling component endogenous to the species of organism from which the cell population is obtained, or a species homologous to the species from which said cell population(s) were obtained, as well as combinations of any two or more thereof. Such signaling molecules include GPCR, activin, BMP, neurotrophic factors, and the like, as well as combinations of any two or more thereof.

Exemplary complex carbohydrates contemplated for use herein include calcium-independent IgSF CAMs (such as, for example, immunoglobulin superfamily CAMs (IgSF CAMs) including homophilic or heterophilic species which bind integrins or different IgSF CAMs; examples of some members of this family include neural cell adhesion molecules (NCAMs), intercellular cell adhesion molecule (ICAM-1), vascular cell adhesion molecule (VCAM-1), Platelet-endothelial Cell Adhesion Molecule (PECAM-1), L1, CHL1, MAG, nectins and nectin-like molecules, and the like); integrins (a family of heterophilic CAMs that bind IgSF CAMs or the extracellular matrix), lymphocyte homing receptors (also known as addressins, including CD34 and GLYCAM-1), and the like); calcium-dependent IgSF CAMs (such as, for example, cadherins (a family of homophilic CAMs, Ca²⁺-dependent, such as E-cadherins (epithelial), P-cadherins (placental), and N-cadherins (neural), selectins (a family of heterophilic CAMs that bind fucosylated carbohydrates, e.g., mucins, including E-selectin, (endothelial), L-selectin (leukocyte), and P-selectin (platelet), and the like, as well as combinations of any two or more thereof.

Exemplary naturally occurring low molecular weight biologically active molecules contemplated for use herein include hormones, retinoic acid, ICCB Known Bioactives Library (BioMol) (see, for example, Pan, H. et. al., J. Biol. Chem. 2008 283 33808; Zhang, L. et. al., PNAS 2007 104 19023; and A. Shelat and K. G. Guy, Nat. Chem. Biol. 2007 3 442); Natural Products Library (BioMol) (see, for example, M. Tulp et al. Drug Discov. Today 2004 9 450; A. Harvey Drug Discov. Today 2000 5 294; D. J. Newman et al. J. Nat. Prod. 2003 66 1022; and M. Heinrich et al. J. Pharm. Pharmacol. 2001 53 425), and the like, as well as combinations of any two or more thereof.

Exemplary synthetic low molecular weight biologically active molecules contemplated for use herein include MaxiVerse™ from Molecular Diversity Libraries (MolBio), LOPAC¹²⁸⁰ (from Sigma), MyriaScreen Diversity Collection of drug-like screening compounds (from Sigma), compound libraries available on the world-wide web from biofocus.com/offerings/compound-libraries.htm?gclid=CMXYzorejp4CFSZdagodhktmsw, and the like, as well as combinations of any two or more thereof.

Additional exemplary naturally occurring and synthetic low molecular weight biologically active molecules contemplated for use herein include antiproliferatives, enzyme inhibitors, cell cycle regulators, apoptosis inducers, GPCR ligands, second messenger modulators, nuclear receptor ligands, actin and tubulin modulators, kinase inhibitors, protease inhibitors, ion channel blockers, gene regulation agents, lipid biosynthesis inhibitors, phosphodiesterase inhibitors, G-Proteins, cyclic nucleotides, multi-drug resistance, neurotransmission inhibitors, phosphatase inhibitors, and the like, as well as combinations of any two or more thereof.

Exemplary polypeptides contemplated for use herein include protein transduction domain (PTD) peptides, and the like, as well as combinations of any two or more thereof.

Exemplary biopolymers contemplated for use herein include polyalkylene oxides, poly(ethylene glycol-co-acryloyl glycolic caproic acid), poly(acryloyl-6-amino caproic acid), poly(acryloyl-2-acrylamido glycolic acid), poly(2-hydroxyethyl methacrylate), poly(N-isopropylacylamide), poly(trimethylene carbonate), poly(acryloyl-4-aminobenzoic acid), poly(acrylamido-methyl-propane sulfonate), poly(3-(methacryloylamino)propyl)dimethyl(3-sulfopropyl)ammonium hydroxide), poly(3-(methacryloylamino)propyl)timethylammonium chloride, poly(ethylene-co-acrylic acid), poly(acrylic acid), poly(L-lactide), poly(D-lactide), poly(DL-lactide-co-glycolide) 85:15, poly(DL-lactide-co-glycolide) 75:25, poly(DL-lactide-co-glycolide) 65:35, poly(DL-lactide-co-caprolactone) 86:14, poly(DL-lactide-co-caprolactone) 40:60, polycaprolactone, poly(3-hydroxybutyric acid-co-3-hydroxyvaleric acid), poly(3-hydroxybutyric acid), polypropylene carbonate), poly(methyl vinyl ether-alt-maleic anhydride), hydrophilic, poly(sodium 4-styrenesulfonate), poly-L-arginine hydrochloride, poly-D-lysine hydrobromide, poly-L-glutamic acid sodium salt, poly-L-ornithine hydrobromide, poly(2-ethyl-2-oxazoline), poly(oligoethylene glycol methyl ethyl methacrylate), poly(butyl methacrylate), poly(ethyl methacrylate), poly(styrene-co-methacrylic acid), poly-L-arginine hydrochloride, poly(ethylene glycol)methacrylate, poly(styrene-alt-maleic acid), poly(styrene), poly(ethylene-alt-maleic anhydride), poly(4-styrenesulfonic acid-co-maleic acid), poly(methyl vinyl ether-alt-maleic acid), poly(methyl vinyl ether), poly(styrene-co-maleic anhydride), poly(isobutylene-co-maleic acid), poly(maleic anhydride-alt-1-octadecene), poly(styrene-alt-maleic anhydride), partial methyl ester, poly(ter-butyl methacrylate), poly(2-hydroxyethyl methacrylate), poly(benzyl methacrylate), poly(2-(dimethylamino)ethyl methacrylate), poly(4-vinylphenol-co-methyl methacrylate), poly(ethylene-co-gylcidyl-methacrylate), poly(cyclohexyl methacrylate), poly(tert-butyl acrylate-co-ethyl acrylate-co-methacryalic acid), poly(ethylene-co-methyl acrylate-co-glycidyl methacrylate), poly(ethylene-co-acrylic acid), poly(ethylene-co-acrylic acid), poly(vinyl alcohol), poly(vinylphosphonic acid), poly(vinyl sulfate) potassium salt, poly(4-vinylpyridine hydrochloride), poly(4-vinylphenol), poly(4-vinylpyridine) crosslinked, poly(vinyl-co-ethylene), poly(vinyl butyral-co-vinyl alcohol-co-vinyl acetate), poly(ethylene-co-vinyl acetate-co-carbon monoxide), poly(allylamine hydrochloride), poly(anetholesulfonic acid), poly(epoxysuccinic acid), poly(1,4-butylene terephthalate), poly(styrene-co-4-bromostyrene-co-divinylbenzene), poly(1,6-hexanediol/neopentyl glycol-alt-adipic acid), poly(acrylonitrile), poly(styrene-co-allyl alcohol), poly(N′,N′-(1,3-phenylene)-isophthalamide), poly(trimellitic anhydride chloride-co-4′,4′-methylene-dianiline), poly(Bisphenol A carbonate), poly(azelaic anhydride), poly(trimethylolpropane di(propylene glycol)-alt-adipic acid/phthalic anhydride), poly(di(ethylene glycol adipate), poly(allyamine), poly(diallyl dimethyl ammonium), poly(diallyl methylamine hydrochloride), poly(1-glyceryl monomethacrylate), poly(3-chroloro-2-hydroxypropyl-2-methacroxyethydimethylammonium chloride), poly(butadienne maleic acid), poly(vinyl pyrroliodone), poly(n-vinylpyrrolidone-vinylacetate), poly(ethylenimine), chitosan, poly(l-glyceryl monomethacrylate), and the like, as well as combinations of any two or more thereof.

Exemplary antibodies contemplated for use herein include any antibody (or fragment thereof) that can functionally interact with human cell types, whether said antibody is monoclonal or polyclonal. Exemplary antibodies include antibodies of the immunoglobulin subtype, Fab fragments, and the like, e.g., antibodies:
which recognize cell surface markers unique to the target cell population;
which recognize any cell surface protein(s) the expression of which is induced by exposure to multi-factorial media;
which inhibit known cell signaling pathways; or
which activate known cell signaling pathways,
   and the like, as well as combinations of any two or more thereof.

Exemplary nucleic acids contemplated for use herein include oligonucleotides, DNA molecules, RNA molecules, and the like, as well as combinations of any two or more thereof.

Exemplary DNA molecules contemplated for use herein include DNA-plasmids/vectors encoding Zinc-finger nucleases, Zinc-finger transcription factors, cDNA over-expression libraries, and the like, as well as combinations of any two or more thereof.

Exemplary RNA molecules contemplated for use herein include siRNA (see, for example, sigmaaldrich.com/life-science/functional-genomics-and-mai/sima.html on the world-wide web), shRNA (see, for example, (sigmaaldrich.com/life-science/functional-genomics-and-mai.html and openbiosystems.com/RNAi/shrnaLibraries/ as available on the world-wide web), microRNA (see, for example, mirbase.org/index.shtml as available on the world-wide web), and the like, as well as combinations of any two or more thereof. As readily recognized by those of skill in the art, RNA molecules can be spotted onto an array either directly (e.g., using siRNA or microRNA), or as a virus containing a viral expression vector containing the RNA molecule of interest (e.g., microRNA or shRNA).

Exemplary lipids contemplated for use herein, or components thereof, include fatty acyls, glycerolipids, glycerophospholipids, sphingolipids, sterol lipids, prenol lipids, saccharolipids, polyketides, and the like, as well as combinations of any two or more thereof.

Exemplary vitamins and metabolites thereof (e.g., retinoic acid is a metabolite of Vitamin A), or functional components thereof, include vitamin A, vitamin B, vitamin C, vitamin D, vitamin E, vitamin K, and the like, as well as combinations of any two or more thereof.

Exemplary inorganic salts contemplated for use herein, or functional components thereof, include calcium chloride (CaCl₂), ferric nitrate (Fe(NO₃), magnesium sulfate (MgSO₄), potassium chloride (KCl), sodium bicarbonate (NaHCO₃), sodium chloride (NaCl), sodium phosphate dibasic (Na₂HPO₄—H₂O), cupric sulfate, manganous chloride, sodium selenite, zinc sulfate (ZnSO₄-7H₂O), sodium phosphate monobasic (NaH₂PO₄—H₂O), magnesium chloride (anhydrous), ferric sulfate (FeSO₄-7H₂O), and the like, as well as combinations of any two or more thereof.

Exemplary media supplements contemplated for use herein, or functional components thereof, include linoleic acid, lipoic Acid, hypoxanthine Na, putrescine 2HCl, sodium pyruvate, thymidine, knock-out serum replacement, glutamine and derivatives thereof, human plasmanate, and the like, as well as combinations of any two or more thereof.

As used herein, reference to a “plurality of complex microenvironments” embraces the use of array technology wherein a substantial number of microenvironments are applied to a single substrate. Typically, in the range of about 200 up to about 250,000 different microarrays are applied to a single substrate. An advantage of the methodology contemplated for use herein is the ability to screen a multitude of possible media formulations with a relatively small number of test cells, typically only about 250,000 cells are required to seed an array comprising in the range of about 200 up to about 250,000 different microenvironments (depending on the seeding level employed (wherein arrays contemplated for use herein comprise anywhere from a single cell per well/spot, up to about 500 cells (or more) per well/spot)). Substrates contemplated for use herein preferably comprise in the range of about 200 up to about 200,000 different microenvironments, with substrates comprising in the range of about 500 up to about 10,000 microenvironments being presently preferred.

In accordance with the present invention, a plurality of complex microenvironments, wherein each complex microenvironment comprises a plurality of the above-described components, are created employing techniques which are well known in the art. For example, glass slides can be cleaned, silanized, and then functionalized with a gel coating (e.g., acrylamide—which is presently preferred because of the non-fouling nature thereof, which facilitates confining test cells to the printed spots on the substrate). Various components contemplated for use in the plurality of microenvironments can then be applied individually or combinatorially to the slides employing techniques which are known in the art, e.g., a commercial arrayer.

In accordance with the present invention, screening of defined cell subpopulation(s) against a plurality of complex microenvironments can be carried out in a variety of ways, e.g., by placing cells of interest on the array slides and allowing them to settle on the spots. Cell media are typically replenished daily. Due to the non-fouling nature of preferred substrates (e.g., acrylamide), test cells are confined substantially to the printed spots. After live imaging, the arrays can then be fixed, and thereafter stained for detection with a specific antibody against one or more markers of choice. Alternatively, cells on the arrays can be stained live for DNA, and thereafter fixed for further screening.

Similarly, in accordance with the present invention, screening of an aberrant cell subpopulation(s) against a plurality of complex microenvironments can be carried out in a variety of ways, e.g., by placing cells of interest on the array slides and allowing them to settle on the spots. Cell media are typically replenished daily. Due to the non-fouling nature of preferred substrates (e.g., acrylamide), test cells are confined substantially to the printed spots. Cells on the arrays can then be stained live for DNA. After live imaging, the arrays can then be fixed, and thereafter stained for detection with a specific antibody against one or more markers of choice.

Exemplary cells can be obtained from a variety of sources, e.g., clinical biopsies, surgical masses, tumor cells being propagated under specific serum free conditions, tumor cells being grown in select niches within mice, and the like.

In accordance with yet another aspect of the present invention, there are provided media formulations identified by any of the screening methods described herein. Such media formulations are useful for a variety of purposes, e.g., for maintaining the viability of a desired subpopulation of cells.

In accordance with a still further aspect of the present invention, there are provided methods for the short- and/or long-term culture of cell populations(s) as defined herein, said methods comprising contacting said cell population with any of the media formulations identified by the methods described herein. One of skill in the art can readily identify contacting conditions suitable for such culturing.

In accordance with another aspect of the present invention, there are provided cell populations propagated by contacting cell subpopulation(s) as defined herein with any of the media formulations identified by the methods described herein. Such cell populations are useful for a variety of purposes, e.g., for testing to evaluate the efficacy of putative drug candidates.

Also provided in accordance with the present invention are methods for identifying media for the in vitro differentiation of cell subpopulations as defined herein, said method comprising:
creating a plurality of complex microenvironments, wherein each complex microenvironment comprises a plurality of components,
screening said cell subpopulation against said plurality of complex micro environments, and
selecting those complex microenvironments which facilitate in vitro differentiation of said cell subpopulation.

As used herein, “differentiation” refers to the process by which a cell is transformed from less specialized to more specialized, or vice versa. For example, unspecialized cells or partially differentiated cells may become more specialized so as to carry out a particular function in the tissues or organs of multicellular organisms. Alternatively, more specialized cells may become de-differentiated to a less specialized state.

Media identified by applying invention methods can be used for a variety of purposes, e.g., for testing putative drug candidates for the ability to promote, or inhibit differentiation of a target cell population.

Also provided in accordance with the present invention are methods for identifying media which induce programmed cell death (apoptosis) of cell subpopulations as defined herein, said method comprising:
creating a plurality of complex microenvironments, wherein each complex microenvironment comprises a plurality of components,
screening said cell subpopulation against said plurality of complex micro environments, and
selecting those complex microenvironments which induce apoptosis of said cell subpopulation.

As used herein, “programmed cell death” (also referred to in the art as “apoptosis”) refers to, i.e., a process involving a series of biochemical events leading to a characteristic cell morphology and death; in more specific terms, a series of biochemical events that lead to a variety of morphological changes, including changes to the cell membrane such as loss of membrane asymmetry and attachment, cell shrinkage, nuclear fragmentation, chromatin condensation, and chromosomal DNA fragmentation.

Media identified by applying invention methods can be used for a variety of purposes, e.g., for testing putative drug candidates for the ability to promote, or inhibit apoptosis of a target cell population.

Also provided in accordance with the present invention are methods for identifying media which promote cell senescence of cell subpopulations as defined herein, said method comprising:
creating a plurality of complex microenvironments, wherein each complex microenvironment comprises a plurality of components,
screening said cell subpopulation against said plurality of complex micro environments, and
selecting those complex microenvironments which promote senescence of said cell subpopulation.

As used herein “cell senescence” refers to the aging of cells. Cellular senescence is the phenomenon by which normal diploid cells lose the ability to divide, normally after about 50 cell divisions in vitro. Some cells become senescent after fewer replications cycles as a result of DNA double strand breaks, toxins etc.

Media identified by applying invention methods can be used for a variety of purposes, e.g., for testing putative drug candidates for the ability to promote, or inhibit senescence of a target cell population.

Also provided in accordance with the present invention are methods for identifying media which modulate the retardation of cell growth of cell subpopulation(s) as defined herein, said method comprising:
creating a plurality of complex microenvironments, wherein each complex microenvironment comprises a plurality of components,
screening said cell subpopulation against said plurality of complex micro environments, and
selecting those complex microenvironments which modulate the retardation of cell growth of said cell subpopulation.

As used herein “modulate the retardation of cell growth” refers to the ability to upregulate or downregulate otherwise suppressed cell growth.

Media identified by applying invention methods can be used for a variety of purposes, e.g., for testing putative drug candidates for the ability to promote, or inhibit senescence of a target cell population.

Also provided in accordance with the present invention are articles comprising a multi-factorial array of components selected from the group consisting of extracellular matrix proteins or components thereof, cellular adhesion molecules, monosaccharides, oligosaccharides, polysaccharides, glycoproteins, proteoglycans, non-proteoglycan polysaccharides, cell communication molecules, complex carbohydrates, lipids, vitamins and metabolites thereof, naturally occurring low molecular weight biologically active molecules, synthetic low molecular weight biologically active molecules, synthetic polymers, biopolymers, antibodies, nucleic acids, inorganic salts, media supplements, and the like, as well as combinations of any two or more thereof.

Invention articles are useful for a variety of purposes, e.g., for identifying compositions which support the culture of defined cell populations; for identifying compositions which promote differentiation of defined cell populations; for identifying compositions which induce apoptosis of defined cell populations; for identifying compositions which promote cell senescence of defined cell populations, and the like.

In accordance with a still further embodiment of the present invention, there are provided methods for screening potentially active agent(s) to identify those which effect a change in one or more properties of an aberrant population of cells, said methods comprising:
creating an array comprising a plurality of spots of a complex microenvironment on which said aberrant population of cells or said potentially active agent(s) are immobilized,
applying potentially active agent(s) or primary cells to each spot on said array such that said array comprises:
   an immobilized aberrant population of cells in contact with said potentially active agent(s), or
   immobilized potentially active agent(s) in contact with said aberrant population of cells,
and evaluating one or more properties of said cells as a function of the potentially active agent(s) with which said cells have been contacted, and
selecting those potentially active agent(s) which effect a change in one or more properties of said aberrant cell population.

Active agents contemplated for use in the practice of the present invention include antibodies, naturally occurring secreted proteins and peptides, soluble receptors, miRNA, siRNA, biosimilars, FAB's, scaffold proteins, viruses (e.g., adenovirus or lentivirus), bacteriophage, other large molecular weight molecules, and the like, as well as combinations of any two or more thereof.

As used herein, antibodies refers to any antibody (or fragment thereof) that can functionally interact with human cell types, whether said antibody is monoclonal or polyclonal. Exemplary antibodies include antibodies of the immunoglobulin subtype, Fab fragments, and the like, e.g., antibodies:
which recognize cell surface markers unique to the aberrant cell population;
which recognize any cell surface protein(s) the expression of which is induced by exposure to multi-factorial media;
which inhibit known cell signaling pathways; or
which activate known cell signaling pathways,
   and the like, as well as combinations of any two or more thereof.

As used herein, naturally occurring secreted proteins and peptides refers to proteins involved in extracellular signaling such as cytokines, growth factors, peptide hormones, and the like.

As used herein, soluble receptors refers to protein molecules that reside in the cytoplasm of a cell, to which one or more specific kinds of signaling molecules may attach. Examples of this class of protein includes many subclasses of soluble cytokine receptors.

As used herein, miRNA refers to short RNA molecules (average of about 22 nucleotides) found in eukaryotic cells. miRNAs bind to complementary sequences on target messenger RNA transcripts (mRNAs), usually resulting in translational repression and gene silencing.

As used herein, siRNA refers to a class of double-stranded RNA molecules (about 20-25 nucleotides in length). siRNA interferes with the expression of a specific gene. siRNAs also act in RNAi-related pathways, e.g., as an antiviral mechanism, in shaping the chromatin structure of a genome, and the like.

As used herein, biosimilars refers to follow-on versions of biologic therapeutic molecules that are made by a different sponsor/mechanism, following expiration of the exclusivity period of the initially approved innovator product.

As used herein, FAB's refers to a portion of the antibody structure where antigens can bind. It is composed of one constant and one variable domain from each heavy and light chain of the antibody.

As used herein, scaffold proteins refers to a class of proteins that interact and/or bind with multiple members of a signaling pathway. Scaffold proteins regulate signal transduction and help localize pathway components (organized in complexes) to specific areas of the cell. Examples of signaling scaffold proteins include receptor tyrosine kinases (e.g., epidermal growth factor receptors and platelet-derived growth factor receptors), and the like.

As used herein, viruses refers to a class of infectious organism that can replicate only inside the living cells of other organisms. Viruses consist of nucleic acid surrounded by a protective coat of proteins. Exemplary viruses contemplated herein include adenovirus, lentivirus, and the like).

As used herein, bacteriophage refers to a type of viruses that infect bacteria.

As used herein, other large molecular weight molecules refers to any type of a wide range of molecules having a molecular weight greater than about 800 Dalton. Examples include synthetic biopolymers, polysaccharides, and the like.

Aberrant cell populations contemplated for use herein include aberrant heart cells, aberrant hepatocytes, aberrant pancreatic cells, aberrant lung cells, aberrant brain cells, hyperproliferative cells, and the like.

Presently preferred aberrant population(s) of cells contemplated for use in the practice of the present invention include primary cells, xenograft-derived samples, neoplastic cells, cells having one or more molecular markers indicative of aberrant behavior, adherent cells, and the like, as well as mixtures of any two or more thereof.

As used herein, neoplastic cells refer to cells which are capable of recapitulating a tumor when transplanted into an animal model.

As used herein, cells having one or more molecular markers indicative of aberrant behavior refer to cells which are indicative of proliferative disorders, autoimmune disorders, and the like.

As used herein, proliferative disorders include cell proliferative disorders such as cancers, mast cell proliferative disorders, and the like.

As used herein, said autoimmune disorders include rheumatoid arthritis, allergic rhinitis, lupus, diabetes, and the like.

Exemplary hyperproliferative cells include:
cell population(s) which are capable of recapitulating a tumor when transplanted into an animal model,
cell population(s) that have tumorigenic activity and display stem cell-like properties;
cell population(s) that have one or more molecular markers indicative of aberrant behavior;
cell population(s) that resist drug treatment upon exposure to one or more agent employed for the treatment of hyperproliferative disorders;
cell population(s) that grow in suspension in the presence of serum-free cell culture medium containing one or more of bFGF, EGF, bovine serum albumin, leukemia inhibitory factor, Neuronal Survival Factor, insulin, human transferring;
and the like;
   as well as combinations of any two or more thereof.

Complex microenvironment(s) contemplated for use in the practice of the present invention comprise two or more components selected from the group consisting of extracellular matrix proteins or components thereof, cellular adhesion molecules, monosaccharides, oligosaccharides, polysaccharides, glycoproteins, proteoglycans, non-proteoglycan polysaccharides, cell communication molecules, complex carbohydrates, lipids, vitamins and metabolites thereof, naturally occurring low molecular weight biologically active molecules, synthetic low molecular weight biologically active molecules, polypeptides, synthetic polymers, biopolymers, antibodies, nucleic acids, inorganic salts, media supplements, and the like.

In accordance with another embodiment of the present invention, there are provided methods for screening potentially active agent(s) to identify those which effect a change in one or more properties of an aberrant population of cells, said method comprising:
applying potentially active agent(s) or primary cells to each spot on an array comprising a plurality of spots of a complex microenvironment on which said aberrant population of cells or said potentially active agent(s) are immobilized such that said array comprises:
   an immobilized aberrant population of cells in contact with said potentially active agent(s), or
   immobilized potentially active agent(s) in contact with said aberrant population of cells,
and evaluating one or more properties of said cells as a function of the potentially active agent(s) with which said cells have been contacted, and
selecting those potentially active agent(s) which effect a change in one or more properties of said aberrant cell population.

In accordance with still another embodiment of the present invention, there are provided methods for screening potentially active agent(s) to identify those which effect a change in one or more properties of an aberrant population of cells, said method comprising:
evaluating, as a function of the potentially active agent(s) with which said cells have been contacted, one or more properties of said cells upon application of potentially active agent(s) or primary cells to each spot on an array comprising a plurality of spots of a complex microenvironment on which said aberrant population of cells or said potentially active agent(s) are immobilized such that said array comprises:
   an immobilized aberrant population of cells in contact with said potentially active agent(s), or
   immobilized potentially active agent(s) in contact with said aberrant population of cells, and
selecting those potentially active agent(s) which effect a change in one or more properties of said aberrant cell population.

In accordance with yet another embodiment of the present invention, there are provided methods for screening potentially active agent(s) to identify those which effect a change in one or more properties of an aberrant population of cells, said method comprising:
selecting those potentially active agent(s) which effect a change in one or more properties of said aberrant cell population,
when said potentially active agent(s) or primary cells are applied to each spot on an array comprising a plurality of spots of a complex microenvironment on which said aberrant population of cells or said potentially active agent(s) are immobilized such that said array comprises:
   an immobilized aberrant population of cells in contact with said potentially active agent(s), or
   immobilized potentially active agent(s) in contact with said aberrant population of cells,
and one or more properties of said cells are evaluated as a function of the potentially active agent(s) with which said cells have been contacted.

In accordance with still another embodiment of the present invention, there are provided methods for screening an aberrant population of cells to identify those which are susceptible to exposure to pharmacologically active agent(s), said methods comprising:
creating an array comprising a plurality of spots of a complex microenvironment on which said aberrant population of cells is immobilized,
applying pharmacologically active agent to each spot on said array and assaying for change(s) in the properties of said cells as a function of exposure thereof to the pharmacologically active agent, and
selecting the pharmacologically active agent(s) which effect a desired change in one or more properties of said aberrant cell population.

In accordance with a further embodiment of the present invention, there are provided methods for screening an aberrant population of cells to identify those which are susceptible to exposure to pharmacologically active agent(s), said method comprising:
applying pharmacologically active agent to each spot on an array comprising a plurality of spots of a complex microenvironment on which said aberrant population of cells is immobilized and assaying for change(s) in the properties of said cells as a function of exposure thereof to the pharmacologically active agent, and
selecting the pharmacologically active agent(s) which effect a desired change in one or more properties of said aberrant cell population.

In accordance with a still further embodiment of the present invention, there are provided methods for screening an aberrant population of cells to identify those which are susceptible to exposure to pharmacologically active agent(s), said method comprising:
selecting the pharmacologically active agent(s) which effect a desired change in one or more properties of said aberrant cell population,
when said array comprising a plurality of spots of a complex microenvironment on which said aberrant population of cells is immobilized is created by applying pharmacologically active agent to each spot on said array and assaying for change(s) in the properties of said cells as a function of exposure thereof to the pharmacologically active agent.

Also provided in accordance with the present invention are articles comprising a support having applied to at least one surface thereof an array comprising a plurality of nano-liter size spots of a complex microenvironment on which potentially active agent(s) are immobilized.

In accordance with one embodiment of the present invention, there are provided articles comprising a support having applied to at least one surface thereof an array comprising a plurality of nano-liter size spots of a complex microenvironment on which aberrant cells are immobilized.

Invention articles comprise a multi-factorial array of components selected from the group consisting of extracellular matrix proteins or components thereof, cellular adhesion molecules, monosaccharides, oligosaccharides, polysaccharides, glycoproteins, proteoglycans, non-proteoglycan polysaccharides, cell communication molecules, complex carbohydrates, lipids, vitamins and metabolites thereof, naturally occurring low molecular weight biologically active molecules, synthetic low molecular weight biologically active molecules, synthetic polymers, biopolymers, antibodies, nucleic acids, inorganic salts, media supplements, and the like, as well as combinations of any two or more thereof.

Invention articles are useful for a variety of purposes, e.g., for identifying compositions which support the culture of aberrant cell populations; for identifying compositions which promote differentiation of aberrant cell populations; for identifying compositions which induce apoptosis of aberrant cell populations; for identifying compositions which promote cell senescence of aberrant cell populations, and the like.

The invention will now be described in greater detail with reference to the following non-limiting examples.

### EXAMPLES

### Example 1

### Reagents and Materials

Fetal bovine serum (FBS), RPMI-1640, penicillin G and streptomycin were purchased from GIBCO/BRL-Invitrogen (Carlsbad, Calif., USA). Nunc Rectangular 4 well plates were purchased from Fisher Scientific. Anti Human CD95 (APO-1/Fas) functional grade antibody was purchased from ebiosceince. Cleaved Caspase 3 (Asp 175) Rabbit monoclonal antibody was purchased from cell signalling. Nunc Rectangular 4 well plates were obtained from Fisher Scientific. Formaldehyde (16%) was purchased from Thermo Scientific.

### Example 2

This example illustrates exemplary protocols for obtaining cells of interest from a suitable source. For example, to identify or enrich for putative tumor initiation cells, primary human tumors are separated into single cells, stained with antibodies specific to marker proteins, and isolated by flow cytometry or magnetic beads.

Alternatively, utilizing mouse models, human tumor initiation cells can be isolated and grown in select niches within mice.

As yet another alternative, tumor initiation cells can be cultured in vitro as spheroids. In this case a tumor is isolated and unsorted cell populations are subjected to specific serum free conditions.

As still another alternative, tumor initiation cells that resist drug treatment upon exposure to one or more agent employed for the treatment of hyperproliferative disorders can be cultured in the presence of one or more such agents.

### Example 3

### Slide Production

This example illustrates an exemplary protocol for fabrication of an array suitable for use in the invention methods. Glass slides (75 mm×25 mm×1 mm) are washed 30 min in a suitable organic solvent (e.g., 100% acetone, 100% methanol, and the like), then 30 min in 100% methanol, and then 10 times in Millipore water (MQH₂O). The slides are then etched one hour in 0.05 N NaOH, rinsed five times with MQH₂O, and dried with filtered compressed air, then baked in an oven (at 65° C.) for 1 hour. The slides are then silanized for one hour in a 2% solution of 3-(trimethoxysilyl)propyl methacrylate in anhydrous toluene, then rinsed in toluene, dried with compressed air, and baked for 15 minutes in an oven (65° C.).

40-100 μL of solution of 10.5% (w/v) acrylamide, 0.55% (w/v) bisacrylamide, 10% (w/v) photoinitiator Irgacure 2959, Ciba Specialty Chemicals I2959 (200 μg/mL in 100% methanol) is placed on a silanized slide and covered with a 75 mm×25 mm cover slip. The slide is then exposed to 1.5 mW/cm² 365-nm ultraviolet A light for 10 min and immersed in MQH₂O for 10 min. The cover slip is then removed, leaving a thin (˜60-75 μm) polyacrylamide gel pad. The polyacrylamide slides are soaked in MQH₂O overnight, and then dried on a hot plate (40° C.) for 10 min.

Stock solutions of components such as ECMP are made in 200 mM acetate, 10 mM EDTA, 40% (v/v) glycerol, and 0.5% (v/v) Triton X-100 in MQH₂O, at pH 4.9. Stock solutions of signaling molecules such as bFGF (Invitrogen, Carlsbad, Calif.), BMP-4 (Invitrogen), retinoic acid (Sigma), and Wnt3a are prepared in a buffer containing 40% [v/v] glycerol, 1% [w/v] CHAPS in PBS). Combinations of different components can be mixed in separate 384-well plates.

In certain embodiments, stock solutions of extracellular matrix (ECM) components (0.01 mg/ml-1 mg/ml), such as Collagen I, III, IV, V, VI, Fibronectin, Laminin, and the like, as well as Hyaluronic Acid, gelatin and BSA (negative control) are mixed 1:1 with 200 mM acetate, 10 mM EDTA, 40% (v/v) glycerol, and 0.5% (v/v) Triton X-100 in MQH₂O, at pH 4.9. Individual and combinations of different components are mixed in 384-well plates. This ECM based array is used to screen for the optimal cell attachment conditions.

Once an optimal ECM is identified, stock solutions of such ECM are mixed together, first with an antibody at different concentrations. Then the ECM and antibody mixtures are mixed 1:1 with 200 mM acetate, 10 mM EDTA, 40% (v/v) glycerol, and 0.5% (v/v) Triton X-100 in MQH₂O, at pH 4.9 or 7.2.

All printings are performed using an arrayer such as SpotArray 24 (Perkin Elmer, Waltham, Mass.) or MicroGrid (Genomic Solution, Ann Arbor, Mich.) at room temperature with 65% relative humidity according to instrument manufacture instructions. To control for variability, each spot can be printed in replicates (e.g., of five spots or replicates of 9 as a 3×3 block of sub-array). Prior to their use, slides are soaked in PBS while being exposed to UVC germicidal radiation in a sterile flow hood for 10 min.

Array slides are stored at 65-75% humidity (in the presence of saturated NaCl solution) at 4° C. An example slide from each batch of printing is incubated with Sypro Ruby overnight at room temperature. On the next day, the slide is de-stained using 10% methanol and 7% acetic acid solution. The slide is then washed several times with MQH₂O water and dried on a rack in the dark. Dried slides are then imaged on an Axon Array Scanner and the image is processed by Genepix software. Spot uniformity is examined to ensure that the protein(s) are printed correctly and that no protein diffusion occurs between spots.

To accommodate imaging using the Cellomics VTI, the nine replicates are printed in 3×3 blocks. Each spot is 400 μm in diameter separated by a center-to-center distance of 550 μm. Each block was separated by 700 μm.

Prior to use, the slides are soaked in PBS while being exposed to UVC germicidal radiation in a sterile hood for 30 min.

### Example 4

This example illustrates an exemplary protocol for seeding cells of interest onto an exemplary array. Cells are placed on the array slides (2.5×10⁵ cells per slide) and allowed to settle on the spots for 18 hours prior to rinsing with the medium one time to remove unattached cells and debris. Cell media are typically replenished daily. Due to the non-fouling nature of the acrylamide gel pad on the slides, cells were confined to the printed spots.

### Example 5

This example illustrates an exemplary protocol for fixing and staining cell arrays. Array slides are washed two times with HBSS or PBS, and then fixed in 4% paraformaldehyde (PFA) for 5 min at 4° C., followed by 10 min at room temperature. Immediately before staining, the cells are permeabilized and blocked with 1% (w/v) BSA and 3% (w/v) milk in TBST for 30 min. Slides are then stained with antibodies generated against cell surface markers, such as CD44+/CD24−, CXCR4+, CD133+, CD138−, CD20, alpha2beta1+, CD44+, EpCam+, Cd166+, LGR5, CD24+ in 1% BSA for 1 hour at room temperature, or overnight at 4° C., washed three times with TBS, and incubated with goat anti-rabbit/mouse Alexa 647 at 1:400 for 1 hour at room temperature. Nucleic acids are stained using the Cy3 equivalent POPO-3 (Invitrogen) for 5 min at room temperature.

### Example 6

This example illustrates an exemplary protocol for analyzing cell arrays according to the present invention. Following fixing and staining as described in the preceding example, the slides are washed three times with TBS, then with water after staining and air-dried immediately before imaging. Imaging of slides is performed using a DNA microarray scanner (e.g., Axon 4000B) at 10-um pixel resolution. The POPO-3 nucleic acid stain (Cy3 equivalent) is imaged using a 543-nm laser excitation and 570-nm emission filter. The Alexa 647 (Cy5 equivalent) is imaged using a 633-nm excitation laser and 670-nm emission filter. Images are then quantified using GenePix software (MDS Analytical Technologies, Sunnyvale, Calif.). Slides can also be imaged using a high content imaging system, such as Cellomic (ThemoFisher).

### Example 7

### Cell Culture

The human acute T cell leukemia Jurkat cell line (ATTC Number TIB-153) was purchased from American Type Culture Collection (ATCC). Cells were cultured in RPMI-1640, supplemented with 10% FBS, 2 mmol/liter glutamin and antibiotics (100 IU/ml of penicillin G) and incubated at 37° C. in a humidified atmosphere of 5% CO₂.

### Example 8

### Experimental Procedure

Jurkat T cells were resuspended at 0.2×10⁶/ml in RPMI 1640 medium containing 10% FBS. Prior to use, slides are placed into a Nunc 4 well rectangle culture plate 1 slide per well with 3 ml PBS. Slides are sterilized by being exposed to UVC germicidal radiation in a sterile laminar flow hood for a minimum of 30 min. Then slides are washed 2× with PBS. 1×10⁶ cells were seeded into each well containing one Microarray slide. Cells were incubated in complete growth medium at 37° C. in 5% CO₂ for 3 h, 6 h, 9 h and 24 h. Each time point was carried out in a replicate of two. After 0 h, 3 h, 6 h, 9 h and 24 h, non adherent, floating cells were removed by media aspiration. Cultures were washed 3× with PBS.

After each incubation period, the slides were fixed with ice-cold 4% para-formaldehyde in PBS (pH 7.4) for 15 min, with the first 5 min at 4° C., and the next 10 min at room temperature (RT).

Slides were then washed 3 x with ice-cold PBS, then 3 ml of fresh PBS was added onto the slides and kept at 4° C. until all slides were ready.

After the last time point was reached, all slides were adjusted to RT and permeabilized with 0.3% Triton X-100, and thereafter the cells were washed with PBS 3× for 5 min.

For blocking, the blocking buffer used comprised 1% BSA, 0.1% Triton X in PBS. Cells were incubated with the blocking buffer for 30 min to block the unspecific binding of the antibodies.

Cells were then incubated with the primary antibody (cleaved Caspase-3) 1:400 in blocking buffer for 1 h at room temperature (RT). The resulting cells were then washed 3× with PBS for 5 min.

Cells were then incubated with secondary antibody 1:1000 in blocking buffer for 1 h at room temperature (RT) protected from the light, and thereafter washed 3× with PBS.

Cells were stained with Hoeshst (1:1000 in PBS) for 5 min protected from the light, and then washed with de-ionized (DI) water 3× and air dried for 3 hours at RT. Slides were kept protected from light and analyzed within 24 h.

For determination of fluorescence using the Cellomics VTI instrument, slides were placed in a Thermo Slideport™ and placed on the Cellomics VTI instrument. Using the Cellomics Calibration Wizard Software tool, 6 form factors were created to identify different geographical areas on a slide in the Slideport™. Each 400 μm spot printed on a slide was imaged at 20×. Channel 1 captured the nuclear intensity of each Jurkat cell and channel 2 captured the Caspase 3 signal. Using the Cellomics Compartmental Analysis Software, the Jurkat cells demonstrating Caspase 3 activation was calculated as a total percentage of the cells on that spot.

A Genepix Array Reader was also used to obtain cell number readouts for the ECMP screen and QC for antibody printing.

### Example 9

### Results

As a preliminary step, an ECMP attachment screen was employed to identify appropriate ECMP's (or combinations thereof) that would promote attachment and therefore immobilize Jurkat cells to specific geographical locations on invention arrays. More than 50 ECMP's and combinations thereof were screened; based on such screening, it was found that fibronectin, when printed alone in a 400 μm spot on a hydrogel coated slide (as described herein) consistently supported attachment of Jurkat cells.

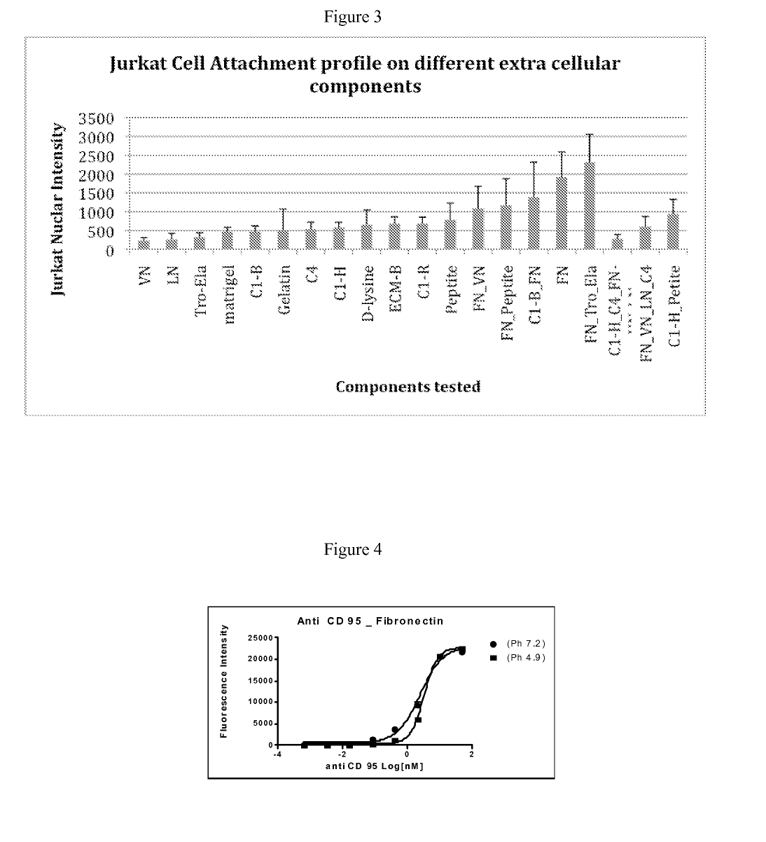

FIG. 3, for example, summarizes the attachment components that were identified to support Jurkat cell attachment on invention arrays. Briefly, Jurkat nuclear intensity was monitored by fluorescence using DraQ staining after 3 hours of attachment. The slides were read using a Genepix DNA microarray and fluorescence was quantified using the Genepix 2.0 software. Each condition represents 9 individual spots seeded with Jurkat cells.

TABLE 1
Individual components screened for Jurkat cell attachment:

| | |
|---|---|
| VN | Vitronectin |
| LN | Laminin |
| Tro-Ela | Tropo elastin |
| matrigel | Matrigel(BD) |
| | Bovine |
| C1-B | Collagen I |
| Gelatin | Gelatin |
| C4 | Collagen IV |
| | Human |
| C1-H | Collagen I |
| D-lysine | Poly-d Lysine |
| FN | Fibronectin |
| | rat Tail |
| C1-R | Collagen I |

After identification of fibronectin as an appropriate ECMP for Jurkat cell capture, anti human CD95 IgM monoclonal antibody was printed in the presence of 250 μg/ml fibronectin at varying concentrations. To ensure retention of the antibody in fibronectin, the antibody was printed in combination with the fibronectin at either pH 7.1 or 4.9. pH was not observed to effect antibody retention.

In order to generate the data presented in FIG. 4, anti CD95 was printed in the presence of fibronectin on hydrogel in 400 μm spots. The antibody was detected using goat anti mouse secondary IgM conjugated to FITC. Slides were imaged on the Genepix DNA array reader and the fluorescent intensity of the spots was quantified. IgM was observed to be retained in the spot regardless of pH.

Jurkat cells immobilized on fibronectin in the presence of anti CD95 demonstrate cell loss over time and Caspase 3 activation. Roughly 500 K Jurkat cells at 100 K cells/ml cell culture media were seeded on the invention array. Cells were cultured on the array for 3-24 hours. Slides were then processed for cell loss or Caspase 3 cleavage using antibody detection of Caspase 3 fragment. Anti-CD95 demonstrated dose dependent activated cell death in Jurkat cells attached to fibronectin.

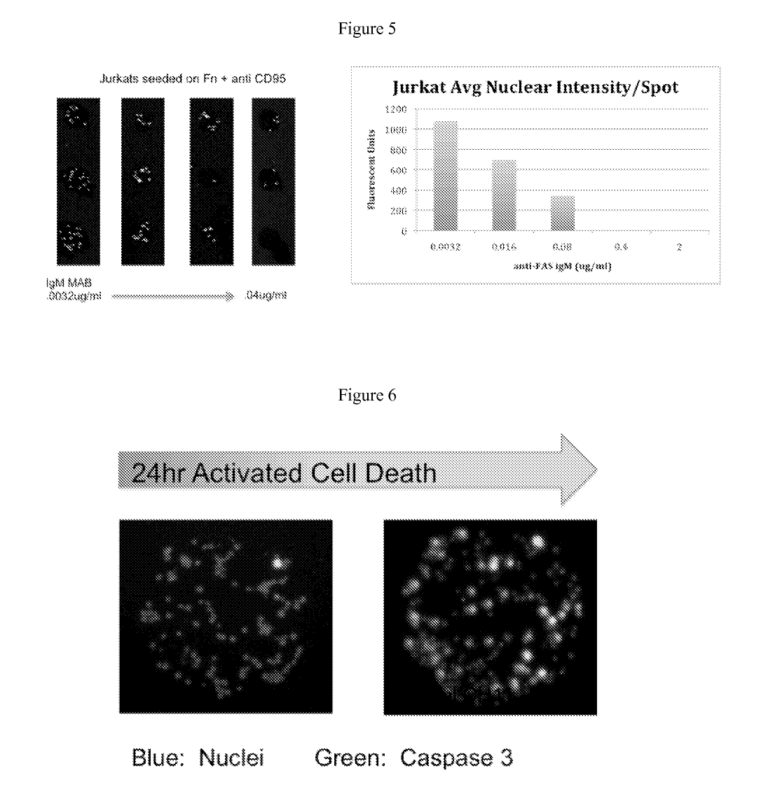

FIG. 5 illustrates that Jurkat cells could not maintain attachment to fibronectin in the presence of anti CD95 IgM MAB. The data summarized in the Figure were generated by incubating Jurkat cells for 48 hrs in the presence of anti CD95. Cell attachment to the spots was observed to be dose dependent.

FIG. 6 demonstrates that Jurkat cells immobilized to fibronectin in the presence of immobilized anti CD95 show an increase in Caspase 3 signal within 24 hr. This Caspase 3 activity is directly correlated to activation of the fas mediated cell death pathway.

The preceding results demonstrate that invention methods and arrays provide a robust platform for screening cells for drug induced functional activity using high content analysis, utilizing physiologically relevant extra cellular matrix proteins to immobilize aberrant cells to geographical locations on a hydrogel coated slide. Within the ECMP it is possible to print (and therefore immobilize) a pharmacologically active agent (e.g., a drug) in such a way that there is sufficient contact with the aberrant cell population. The immobilized cells can be fixed and stained using standard immunofluorescence techniques, thereby enabling in depth high content analysis. The present invention, therefore, provides a highly relevant and robust drug screening system based on the use of ECMP's for natural attachment of aberrant cells. The platform places a pharmacologically active agent (e.g., a drug) in direct contact with aberrant cells ensuring proper exposure thereof (in contrast to the addition of test compounds in solution, whereby the interaction between the components of interest may change).

Although the invention has been described with reference to embodiments and examples, it should be understood that various modifications can be made without departing from the spirit of the invention.

All references cited herein are hereby expressly incorporated by reference in their entireties. Where reference is made to a uniform resource locator (URL) or other such identifier or address, it is understood that such identifiers can change and particular information on the internet can be added, removed, or supplemented, but equivalent information can be found by searching the internet. Reference thereto evidences the availability and public dissemination of such information.

1. A method for identifying media for:
the short- and/or long-term in vitro culture of cell subpopulation(s), or
the in vitro differentiation of cell subpopulations, or
identifying media which induce programmed cell death (apoptosis) of cell subpopulation(s), or
identifying media which promote cell senescence of cell subpopulation(s), or
identifying media which modulate the retardation of cell growth of cell subpopulation(s),
wherein said cell subpopulation(s) are selected from the group consisting of:
cell subpopulation(s) which are capable of recapitulating a tumor when transplanted into an animal model;
cell subpopulation(s) that have tumorigenic activity and display stem cell-like properties;
cell subpopulation(s) that have one or more molecular markers indicative of aberrant behavior;
cell subpopulation(s) that resist drug treatment upon exposure to one or more agent employed for the treatment of hyperproliferative disorders; and
cell subpopulation(s) that grow in suspension in the presence of serum-free cell culture medium containing one or more of bFGF, EGF, bovine serum albumin, leukemia inhibitory factor, Neuronal Survival Factor, insulin, human transferrin;
as well as combinations of any two or more thereof,
said method comprising:
creating a plurality of complex microenvironments, wherein each complex microenvironment comprises a plurality of components,
screening said cell subpopulation(s) against said plurality of complex microenvironments, and
selecting those complex microenvironments which facilitate short-term and/or long-term in vitro culture of said cell subpopulation(s) and/or the in vitro differentiation of cell subpopulations and/or induce programmed cell death (apoptosis) of cell subpopulation(s) and/or promote cell senescence of cell subpopulation(s) and/or modulate the retardation of cell growth of cell subpopulation(s).

2. The method of claim 1, wherein one or more of the plurality of components which comprise each complex microenvironment closely resemble component(s) found in the in vivo environment in which said cell subpopulation(s) normally reside(s), or in the in vivo environment of a species homologous to the species from which said cell population(s) were obtained.

3. The method of claim 1, wherein said plurality of microenvironments comprise a multi-factorial array of two or more components selected from the group consisting of extracellular matrix proteins or components thereof, cellular adhesion molecules, monosaccharides, oligosaccharides, polysaccharides, glycoproteins, proteoglycans, non-proteoglycan polysaccharides, cell communication molecules, complex carbohydrates, lipids, vitamins and metabolites thereof, naturally occurring low molecular weight biologically active molecules, synthetic low molecular weight biologically active molecules, polypeptides, synthetic polymers, biopolymers, antibodies, nucleic acids, inorganic salts, and media supplements.

4. The method of claim 3, wherein said cell communication molecules are selected from the group consisting of growth factors, signaling molecules, hormones and cytokines.

5. The method of claim 1 wherein said cell subpopulation is capable of recapitulating a tumor when transplanted into an animal model.

6. The method of claim 1 wherein said cell subpopulation has tumorigenic activity and displays stem cell-like properties.

7. The method of claim 1 wherein said cell subpopulation has one or more molecular markers indicative of aberrant behavior.

8. The method of claim 7 wherein said cells that have one or more molecular markers indicative of aberrant behavior are selected from the group consisting of cells expressing CD44+/CD24−, CXCR4+, CD133+, CD138−, CD20, alpha2beta1+, CD44+, EpCam+, Cd 166+, LGR5, and CD24+, as well as combinations of any two or more thereof.

9. The method of claim 1 wherein said cell subpopulation resists drug treatment upon exposure to one or more agent employed for the treatment of hyperproliferative disorders.

10. The method of claim 9 wherein said agent employed for the treatment of hyperproliferative disorders is selected from the group consisting of Arabinosylcytosine (ARA-C), Cytarabine, Bleomycin, Busulfan, Capecitabine, Carboplatin, Carmustine, Chlorambucil, Cisplatin, Cyclophosphamide, Dacarbazine, Daunorubicin, Docetaxel, Doxorubicin, Epirubicin, Etoposide, Fludarabine, 5-Fluorouracil, Gemcitabine, Hydroxyurea, Idarubicin, Ifosfamide, Irinotecan, Lomustine, Mechlorethamine, Melphalan, 6-Mercaptopurine (6-MP), Methotrexate, Mitomycin, C-Mitoxantrone, Oxailplatin, Paclitaxel, Streptozocin, Temozolomide, 6-Thioguanine, Topotecan, Vinblastine, Vincristine, Vindesine, Vinorelbine, Alemtuzumab, Bevacizumab, Gemtuzumab, Ibritumomab, Rituximab, Tositumomab, Trastuzumab, Aldesleukin, IL-2, Alpha Interferon, Imiquimod, Lenalidomide, Anastrozole, Bicalutamide, Exemestane, Flutamide, Fulvestrant, Letrozole, Megestrol, Raloxifene, Tamoxifen, and Toremifene.

11. The method of claim 1 wherein said cell subpopulation grows in suspension in the presence of serum-free cell culture medium containing one or more of bFGF, EGF, bovine serum albumin, leukemia inhibitory factor, Neuronal Survival Factor, insulin, and human transferrin.

12. The method of claim 11 wherein said cells that grow in the presence of serum-free cell culture medium containing one or more of bFGF, EGF, bovine insulin, and human transferrin grow as spheroids in suspension culture, and are capable of differentiating into adherent cells in the presence of fetal bovine serum.

13. A method for identifying media for the short-and/or long-term in vitro culture of cell subpopulation(s), wherein said cell subpopulation(s) are selected from the group consisting of:
cell subpopulation(s) which are capable of recapitulating a tumor when transplanted into an animal model;
cell subpopulation(s) that have tumorigenic activity and display stem cell-like properties;
cell subpopulation(s) that have one or more molecular markers indicative of aberrant behavior;
cell subpopulation(s) that resist drug treatment upon exposure to one or more agent employed for the treatment of hyperproliferative disorders; and
cell subpopulation(s) that grow in suspension in the presence of serum-free cell culture medium containing one or more of bFGF, EGF, bovine serum albumin, leukemia inhibitory factor, Neuronal Survival Factor, insulin, human transferrin;
as well as combinations of any two or more thereof,
said method comprising:
screening said cell population(s) against a plurality of complex microenvironments, wherein each complex microenvironment comprises a plurality of components, one or more of which closely resemble or mimic component(s) found in the in vivo environment in which the cell normally resides, and
selecting those microenvironments which facilitate short- and/or long-term culture of said cell population(s).

14. A method for identifying media for the short-and/or long-term culture of cell subpopulation(s), wherein said cell subpopulation(s) are selected from the group consisting of:
cell subpopulation(s) which are capable of recapitulating a tumor when transplanted into an animal model;
cell subpopulation(s) that have tumorigenic activity and display stem cell-like properties;
cell subpopulation(s) that have one or more molecular markers indicative of aberrant behavior;
cell subpopulation(s) that resist drug treatment upon exposure to one or more agent employed for the treatment of hyperproliferative disorders; and
cell subpopulation(s) that grow in suspension in the presence of serum-free cell culture medium containing one or more of bFGF, EGF, bovine serum albumin, leukemia inhibitory factor, Neuronal Survival Factor, insulin, human transferrin;
as well as combinations of any two or more thereof,
said method comprising selecting those microenvironments which facilitate short- and/or long-term culture said cell population(s) when said cell population(s) are screened against a plurality of complex microenvironments,
wherein each complex microenvironment comprises a plurality of components, one or more of which closely resemble component(s) found in the in vivo environment in which the cell subpopulation(s) normally reside(s).

15. Media formulations identified by the method of claim 1.

16. A method for the short- and/or long-term culture of cell populations(s), wherein said cell subpopulation(s) are selected from the group consisting of:
cell subpopulation(s) which are capable of recapitulating a tumor when transplanted into an animal model;
cell subpopulation(s) that have tumorigenic activity and display stem cell-like properties;
cell subpopulation(s) that have one or more molecular markers indicative of aberrant behavior;
cell subpopulation(s) that resist drug treatment upon exposure to one or more agent employed for the treatment of hyperproliferative disorders; and
cell subpopulation(s) that grow in suspension in the presence of serum-free cell culture medium containing one or more of bFGF, EGF, bovine serum albumin, leukemia inhibitory factor, Neuronal Survival Factor, insulin, human transferrin;
as well as combinations of any two or more thereof,
said method comprising contacting said cell population with the media formulation of claim 15.

17. Cell populations propagated by the method of claim 16.

18.-21. (canceled)

22. An article comprising a multi-factorial array of components selected from the group consisting of extracellular matrix proteins or components thereof, cellular adhesion molecules, monosaccharides, oligosaccharides, polysaccharides, glycoproteins, proteoglycans, non-proteoglycan polysaccharides, cell communication molecules, complex carbohydrates, lipids, vitamins and metabolites thereof, naturally occurring low molecular weight biologically active molecules, synthetic low molecular weight biologically active molecules, polypeptides, synthetic polymers, biopolymers, antibodies, nucleic acids, inorganic salts, and media supplements, as well as combinations of any two or more thereof.

23. A method for screening potentially active agent(s) to identify those which effect a change in one or more properties of an aberrant population of cells, said method comprising:
creating an array comprising a plurality of spots of a complex microenvironment on which said aberrant population of cells or said potentially active agent(s) are immobilized,
applying potentially active agent(s) or primary cells to each spot on said array such that said array comprises:
an immobilized aberrant population of cells in contact with said potentially active agent(s), or
immobilized potentially active agent(s) in contact with said aberrant population of cells,
and evaluating one or more properties of said cells as a function of the potentially active agent(s) with which said cells have been contacted, and
selecting those potentially active agent(s) which effect a change in one or more properties of said aberrant cell population.

24. The method of claim 23 wherein said active agents are antibodies, naturally occurring secreted proteins and peptides, soluble receptors, miRNA, siRNA, biosimilars, FAB's, scaffold proteins, viruses (e.g., adenovirus or lentivirus), bacteriophage, or other large molecular weight molecules.

25. The method of claim 23 wherein said aberrant population of cells is selected from the group consisting of primary cells, xenograft-derived samples, neoplastic cells, cells having one or more molecular markers indicative of aberrant behavior, and adherent cells.

26. The method of claim 25 wherein said neoplastic cells are capable of recapitulating a tumor when transplanted into an animal model.

27. The method of claim 25 wherein said cells having one or more molecular markers indicative of aberrant behavior are indicative of proliferative disorders, or autoimmune disorders.

28. The method of claim 27 wherein said proliferative disorders are selected from the group consisting of cell proliferative disorders and mast cell proliferative disorders.

29. The method of claim 27 wherein said autoimmune disorders are selected from the group consisting of rheumatoid arthritis, allergic rhinitis, lupus, and diabetes.

30. The method of claim 23 wherein said complex microenvironment comprises two or more components selected from the group consisting of extracellular matrix proteins or components thereof, cellular adhesion molecules, monosaccharides, oligosaccharides, polysaccharides, glycoproteins, proteoglycans, non-proteoglycan polysaccharides, cell communication molecules, complex carbohydrates, lipids, vitamins and metabolites thereof, naturally occurring low molecular weight biologically active molecules, synthetic low molecular weight biologically active molecules, polypeptides, synthetic polymers, biopolymers, antibodies, nucleic acids, inorganic salts, and media supplements.

31. A method for screening potentially active agent(s) to identify those which effect a change in one or more properties of an aberrant population of cells, said method comprising:
applying potentially active agent(s) or primary cells to each spot on an array comprising a plurality of spots of a complex microenvironment on which said aberrant population of cells or said potentially active agent(s) are immobilized such that said array comprises:
an immobilized aberrant population of cells in contact with said potentially active agent(s), or
immobilized potentially active agent(s) in contact with said aberrant population of cells,
and evaluating one or more properties of said cells as a function of the potentially active agent(s) with which said cells have been contacted, and
selecting those potentially active agent(s) which effect a change in one or more properties of said aberrant cell population, or
evaluating, as a function of the potentially active agent(s) with which said cells have been contacted, one or more properties of said cells upon application of potentially active agent(s) or primary cells to each spot on an array comprising a plurality of spots of a complex microenvironment on which said aberrant population of cells or said potentially active agent(s) are immobilized such that said array comprises:
an immobilized aberrant population of cells in contact with said potentially active agent(s), or
immobilized potentially active agent(s) in contact with said aberrant population of cells, and
selecting those potentially active agent(s) which effect a change in one or more properties of said aberrant cell population, or
selecting those potentially active agent(s) which effect a change in one or more properties of said aberrant cell population,
when said potentially active agent(s) or primary cells are applied to each spot on an array comprising a plurality of spots of a complex microenvironment on which said aberrant population of cells or said potentially active agent(s) are immobilized such that said array comprises:
an immobilized aberrant population of cells in contact with said potentially active agent(s), or
immobilized potentially active agent(s) in contact with said aberrant population of cells,
and one or more properties of said cells are evaluated as a function of the potentially active agent(s) with which said cells have been contacted.

32.-33. (canceled)

34. A method for screening an aberrant population of cells to identify those which are susceptible to exposure to pharmacologically active agent(s), said method comprising:
creating an array comprising a plurality of spots of a complex microenvironment on which said aberrant population of cells is immobilized,
applying pharmacologically active agent to each spot on said array and assaying for change(s) in the properties of said cells as a function of exposure thereof to the pharmacologically active agent, and
selecting the pharmacologically active agent(s) which effect a desired change in one or more properties of said aberrant cell population, or
applying pharmacologically active agent to each spot on an array comprising a plurality of spots of a complex microenvironment on which said aberrant population of cells is immobilized and assaying for change(s) in the properties of said cells as a function of exposure thereof to the pharmacologically active agent, and
selecting the pharmacologically active agent(s) which effect a desired change in one or more properties of said aberrant cell population, or
selecting the pharmacologically active agent(s) which effect a desired change in one or more properties of said aberrant cell population,
when said array comprising a plurality of spots of a complex microenvironment on which said aberrant population of cells is immobilized is created by applying pharmacologically active agent to each spot on said array and assaying for change(s) in the properties of said cells as a function of exposure thereof to the pharmacologically active agent.

35.-36. (canceled)

37. An article comprising a support having applied to at least one surface thereof an array comprising a plurality of nano-liter size spots of a complex microenvironment on which potentially active agent(s) and/or aberrant cells are immobilized.

38. (canceled)
